(19) United States
(12) Patent Application Publication
(10) Pub. No.: US 2025/0295887 A1
(43) Pub. Date: Sep. 25, 2025

(54) CATHETER

(71) Applicant: ASAHI INTECC CO., LTD., Seto-shi (JP)

(72) Inventor: Saisamphan WIJAIRAK, Seto-shi (JP)

(73) Assignee: ASAHI INTECC CO., LTD., Seto-shi (JP)

(21) Appl. No.: 19/227,617

(22) Filed: Jun. 4, 2025

Related U.S. Application Data
(63) Continuation of application No. PCT/JP2023/032854, filed on Sep. 8, 2023.

(30) Foreign Application Priority Data
Dec. 13, 2022 (JP) 2022-198605

(51) Int. Cl. A61M 25/00; A61M 25/01

(52) U.S. Cl. CPC A61M 25/0023; A61M 25/0068; A61M 25/0108; A61M 2205/32

(57) ABSTRACT

A catheter includes a hollow shaft, a distal end side marker that is provided at a distal end portion of the shaft, a rear end side marker that is provided at the distal end portion of the shaft and is closer to a rear end side than the distal end side marker, a distal end side tube covering an outer periphery of the distal end side marker, and a rear end side tube covering an outer periphery of the rear end side marker, and a rear end of the distal end side tube and a distal end of the rear end side tube are provided to be separated from each other.

### CROSS-REFERENCE TO RELATED APPLICATIONS

The present application is a bypass continuation of International Application No. PCT/JP2023/032854, filed Sep. 8, 2023, which claims priority to Japanese patent application JP 2022-198605, filed Dec. 13, 2022, the entire contents of each of which being incorporated herein by reference.

### TECHNICAL FIELD

The present disclosure relates to a catheter.

### BACKGROUND ART

There is a conventionally known method for treating an aneurysm using a catheter. Patent Literature 1 describes a catheter including two markers at a distal end portion, which can be visually recognized by an operator under X-ray fluoroscopy.

### CITATION LIST

### Patent Literature

Patent Literature 1: JP 2015-77305 A

### SUMMARY

### Technical Problems

The distal end portion of the catheter is required to have flexibility for facilitating advancement in a blood vessel.

One or more embodiments are directed to improving the flexibility of a distal end portion of a catheter.

### Solutions to Problems

The present disclosure has been made to solve at least a part of the issue described above and can be implemented as the following aspects.

(1) According to an aspect of the present disclosure, a catheter includes a hollow shaft, a distal end side marker at a distal end portion of the shaft, a rear end side marker at the distal end portion of the shaft closer to a rear end side than the distal end side marker, a distal end side tube covering an outer periphery of the distal end side marker, and a rear end side tube covering an outer periphery of the rear end side marker, and a rear end of the distal end side tube and a distal end of the rear end side tube are separated from each other.

With this configuration, since the distal end side tube and the rear end side tube are separated from each other, the flexibility of the distal end portion of the catheter can be improved.

The present disclosure can be implemented in various aspects and can be implemented in the form of, for example, a guide wire, a method for manufacturing a guide wire, a catheter, a method for manufacturing a catheter, an endoscope, or a dilator.

### BRIEF DESCRIPTION OF DRAWINGS

FIG. 1 is an explanatory view illustrating an overall configuration of a catheter according to a first embodiment.

FIG. 2 is an explanatory view illustrating a longitudinal section of a distal end portion of the catheter.

FIG. 3 is an explanatory view illustrating a longitudinal section in the vicinity of a marker.

FIG. 4 is an explanatory view illustrating a transverse section of the catheter.

FIG. 5 is an explanatory view illustrating a longitudinal section in the vicinity of a marker of a catheter according to a second embodiment.

FIG. 6 is an explanatory view illustrating a longitudinal section in the vicinity of a marker of a catheter according to a third embodiment.

FIG. 7 is an explanatory view illustrating a longitudinal section in the vicinity of a marker of a catheter according to a fourth embodiment.

FIG. 8 is an explanatory view illustrating a longitudinal section in the vicinity of a marker of a catheter according to a fifth embodiment.

### DETAILED DESCRIPTION

### First Embodiment

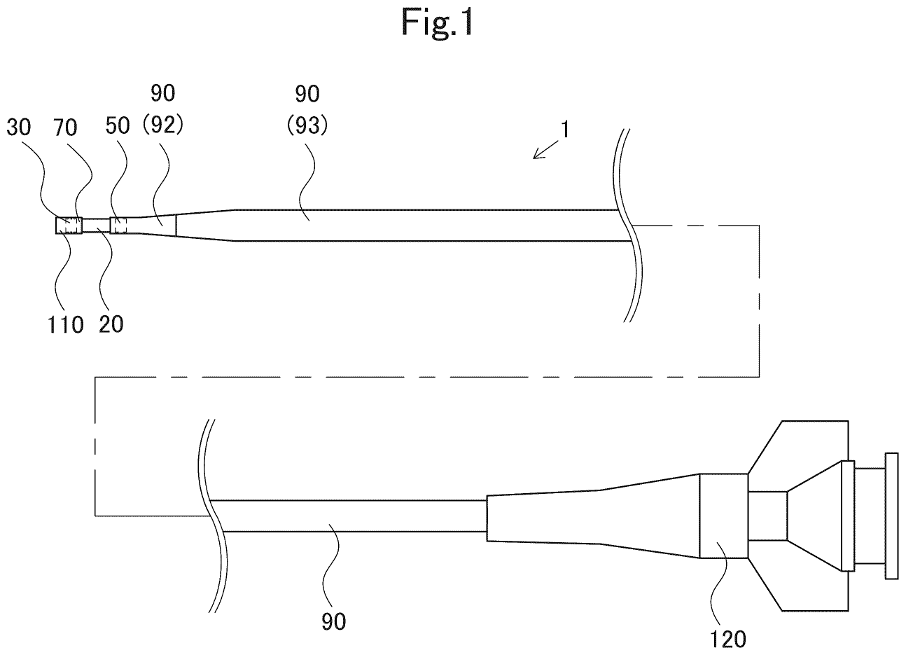

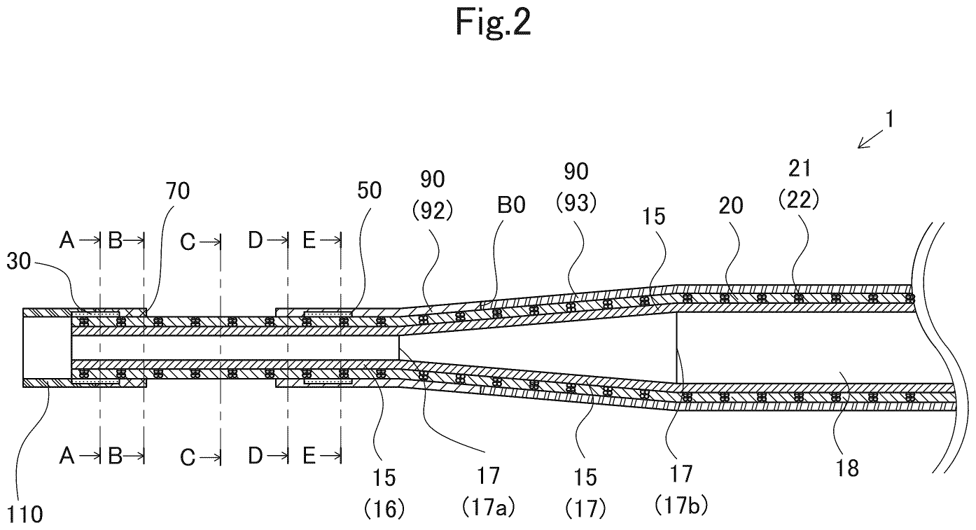

FIG. 1 is an explanatory view illustrating the overall configuration of a catheter 1 according to a first embodiment. FIG. 2 is an explanatory view illustrating a longitudinal section of a distal end portion of the catheter 1. Hereinafter, an end portion located on the distal end side of each constituent member of the catheter 1 will be referred to as a “distal end”, and a portion that includes the “distal end” and extends halfway from the distal end toward the rear end side will be referred to as a “distal end portion”. Similarly, an end portion located on the rear end side of each constituent member is referred to as a “rear end”, and a portion that includes the “rear end” and extends halfway from the rear end toward the distal end side is referred to as a “rear end portion”.

The catheter 1 is a medical device that is inserted into a blood vessel, a digestive organ, or the like, and is used for treatment, diagnosis, or the like, of a human body.

As illustrated in FIGS. 1 and 2, the catheter 1 includes a shaft 15 (FIG. 2), an intermediate tube 20, a distal end side tube 70, a rear end side tube 90, a distal end side marker 30, a rear end side marker 50, a distal tip 110, and a connector 120 (FIG. 1).

As illustrated in FIG. 2, the shaft 15 is a hollow member extending in the longitudinal direction of the catheter 1. The shaft 15 includes a straight portion 16 and a tapered portion 17. The straight portion 16 is a portion of the shaft 15 having a substantially constant outer diameter in the longitudinal direction of the shaft 15. As used herein, “substantially” is to indicate within manufacturing tolerances and is used to indicate approximation, not as a relative term, and may be applicable to any descriptive parameter, e.g., straight, equal, and so forth, whether or not the term is used in connection with the descriptive parameter. The tapered portion 17 is a portion of the shaft 15 in which the outer diameter increases from the distal end side toward the rear end side of the shaft 15. The outer diameter of a rear end 17b of the tapered portion 17 is larger than the outer diameter of a distal end 17a of the tapered portion 17. According to the present embodiment, the straight portion 16 is provided on the distal end side of the shaft 15, and the tapered portion 17 is provided closer to the rear end side than the straight portion 16. A lumen 18 is formed inside the shaft 15. The lumen 18 is a space that is surrounded by the inner periphery of the shaft 15 and extends in the longitudinal direction of the shaft 15. A guide wire, a delivery device for delivering an embolic substance used for treatment of an aneurysm, or the like, can be passed through the lumen 18.

The intermediate tube 20 is a hollow member covering the outer periphery of the shaft 15. A braid 21 is provided inside the intermediate tube 20 along the longitudinal direction of the catheter 1. In the example shown, the braid 21 is formed by braiding four wires 22.

The distal end side tube 70 is a hollow member covering a part of the outer periphery of the intermediate tube 20 and a part of the outer periphery of the distal end side marker 30. The distal end side tube 70 is provided on the outer periphery of the straight portion 16 of the shaft 15.

The rear end side tube 90 is a hollow member covering a part of the outer periphery of the intermediate tube 20 and the outer periphery of the rear end side marker 50. The rear end side tube 90 is provided closer to the rear end side than the distal end side tube 70 in the longitudinal direction of the shaft 15. The rear end side tube 90 includes a first rear end side tube 92 and a second rear end side tube 93. The first rear end side tube 92 covers the outer periphery of the distal end side of the tapered portion 17 of the shaft 15. The second rear end side tube 93 covers the outer periphery of the rear end side of the tapered portion 17 of the shaft 15. A boundary between the first rear end side tube 92 and the second rear end side tube 93 is referred to as a “boundary B0”.

The boundary B0 is between the distal end 17a and the rear end 17b of the tapered portion 17 of the shaft 15 and is closer to the distal end side than the intermediate position of the tapered portion 17 where the distances from the distal end 17a and the rear end 17b are equal. The bending rigidity of the first rear end side tube 92 is smaller than the bending rigidity of the second rear end side tube 93. Therefore, in the rear end side tube 90, the bending rigidity of the portion covering the distal end side of the tapered portion 17 is smaller than the bending rigidity of the portion covering the proximal end side. The methods for measuring the bending rigidity of the portion of the rear end side tube 90 covering the distal end side of the tapered portion 17 and the bending rigidity of the portion covering the proximal end side are as follows.

### (1) Bending Rigidity of the Portion Covering the Distal End Side of the Tapered Portion 17

The mean value of the bending rigidity of the rear end side tube 90 at each position of the rear end side tube 90 obtained by equally dividing the portion between the distal end 17a and the intermediate position of the tapered portion 17 into four portions at equal intervals.

### (2) Bending Rigidity of the Portion Covering the Proximal End Side of the Tapered Portion 17

The mean value of the bending rigidity of the rear end side tube 90 at each position of the rear end side tube 90 obtained by equally dividing the portion between the intermediate position and the rear end 17b of the tapered portion 17 into four portions at equal intervals.

The difference in the bending rigidity between the first rear end side tube 92 and the second rear end side tube 93 can be set, for example, by forming the two tubes from materials having different hardnesses or by changing the thicknesses of the two tubes.

The respective materials of the shaft 15, the intermediate tube 20, the distal end side tube 70, and the rear end side tube 90 are not particularly limited, and it is possible to use, for example, nylon resin such as polyamide, polyethylene, polypropylene, polyolefin such as ethylene-propylene copolymers, polyester such as polyethylene terephthalate, polyvinyl chloride, ethylene-vinyl acetate copolymers, crosslinked ethylene-vinyl acetate copolymer, thermoplastic resin such as polyurethane, fluororesin such as polytetrafluoroethylene (PTFE), polyamide elastomer, polyolefin elastomer, polyurethane elastomer, silicone rubber, latex rubber, etc.

The distal end side marker 30 is an annular member made of a material having a low X-ray transmittance. The distal end side marker 30 is provided at the distal end portion of the shaft 15. Specifically, the distal end side marker 30 is provided on the outer periphery of the straight portion 16 of the shaft 15. The phrase “the distal end side marker 30 is provided at the distal end portion of the shaft 15” means that at least a part of the distal end side marker 30 is at the same position in the axial direction as the distal end portion of the shaft 15, and the distal end side marker 30 does not necessarily need to be directly connected to the distal end portion of the shaft 15. Here, the distal end side marker 30 is indirectly fixed to the distal end portion of the shaft 15 via the intermediate tube 20. The outer periphery of the distal end portion of the distal end side marker 30 is covered with the distal tip 110, and the outer periphery of the rear end portion of the distal end side marker 30 is covered with the distal end side tube 70.

The rear end side marker 50 is an annular member made of a material having a low X-ray transmittance. The rear end side marker 50 is provided at the distal end portion of the shaft 15. Specifically, the rear end side marker 50 is provided on the outer periphery of the straight portion 16 of the shaft 15. The phrase “the rear end side marker 50 is provided at the distal end portion of the shaft 15” means that at least a part of the rear end side marker 50 is at the same position in the axial direction as the distal end portion of the shaft 15, and the rear end side marker 50 does not necessarily need to be directly connected to the distal end portion of the shaft 15. Here, the rear end side marker 50 is indirectly fixed to the distal end portion of the shaft 15 via the intermediate tube 20. The rear end side marker 50 is closer to the rear end side than the distal end side marker 30. The outer periphery of the rear end side marker 50 is covered with the rear end side tube 90.

The shapes of the distal end side marker 30 and the rear end side marker 50 are not limited to an annular shape and may be, for example, a plate shape covering only a part of the outer periphery of the intermediate tube 20, or a coil shape as described below in a third embodiment.

The material of the distal end side marker 30 and the material of the rear end side marker 50 are not particularly limited, and it is possible to use, for example, gold, platinum, tungsten, or an alloy containing these elements (for example, a platinum-nickel alloy). Alternatively, as the material of the distal end side marker 30 and the material of the rear end side marker 50, it is possible to use, for example, a resin material formed by mixing a radiopaque material such as bismuth trioxide, tungsten, or barium sulfate with a polyamide resin, a polyolefin resin, a polyester resin, a polyurethane resin, a silicone resin, or a fluororesin.

The distal tip 110 is a hollow member having a space that communicates with the lumen 18 of the shaft 15. The distal tip 110 is connected to the distal end of the shaft 15, the distal end of the intermediate tube 20, the distal end of the distal end side tube 70, and the distal end of the distal end side marker 30. The rear end portion of the distal tip 110 covers the outer periphery of the distal end portion of the distal end side marker 30.

The material of the distal tip 110 is not particularly limited, and it is possible to use, for example, a resin material such as polyurethane, polyurethane elastomer, polyamide, or polyamide elastomer.

The connector 120 is a member that connects the catheter 1, which is connected to the rear end portion of the rear end side tube 90, to another medical device such as a syringe.

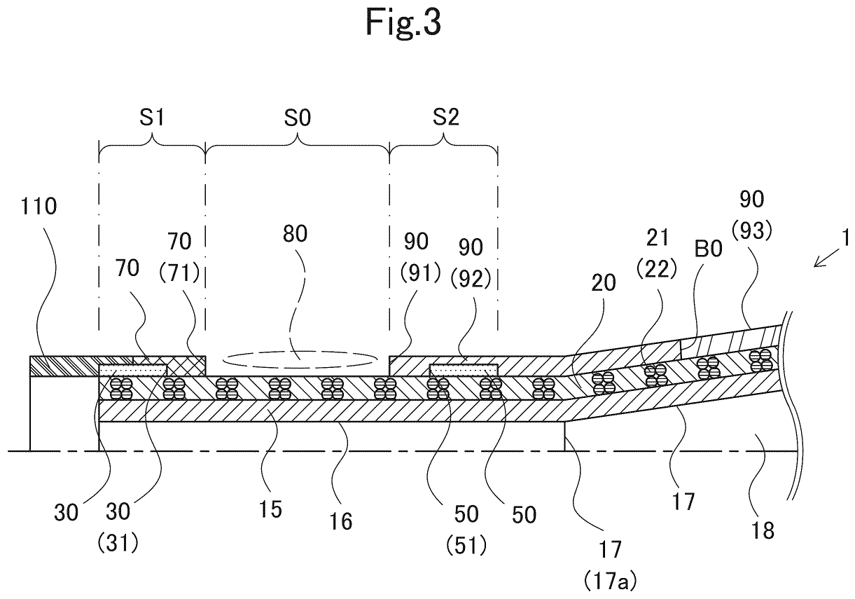

FIG. 3 is an explanatory view illustrating a longitudinal section of the vicinity of the markers (30, 50) in the catheter 1. As described above, the distal end side tube 70 covers a part of the outer periphery of the distal end side marker 30. A rear end 71 of the distal end side tube 70 is closer to the rear end side than a rear end 31 of the distal end side marker 30. Thus, the outer periphery of the distal end side marker 30 is not exposed to the outside. The rear end side tube 90 covers the outer periphery of the rear end side marker 50. A distal end 91 of the rear end side tube 90 is closer to the distal end side than a distal end 51 of the rear end side marker 50. Thus, the outer periphery of the rear end side marker 50 is not exposed to the outside.

The rear end 71 of the distal end side tube 70 and the distal end 91 of the rear end side tube 90 are provided to be separated from each other. In the catheter 1, the section between the rear end 71 of the distal end side tube 70 and the distal end 91 of the rear end side tube 90 is referred to as a “section S0”. The space between the rear end 71 of the distal end side tube 70 and the distal end 91 of the rear end side tube 90, which is provided at the section S0, is referred to as a “gap 80”. The gap 80 is formed along the outer periphery of the intermediate tube 20. In the catheter 1, the section from the distal end of the distal end side marker 30 to the rear end 71 of the distal end side tube 70 is referred to as a “section S1”. In the catheter 1, the section from the rear end of the rear end side marker 50 to the distal end 91 of the rear end side tube 90 is referred to as a “section S2”.

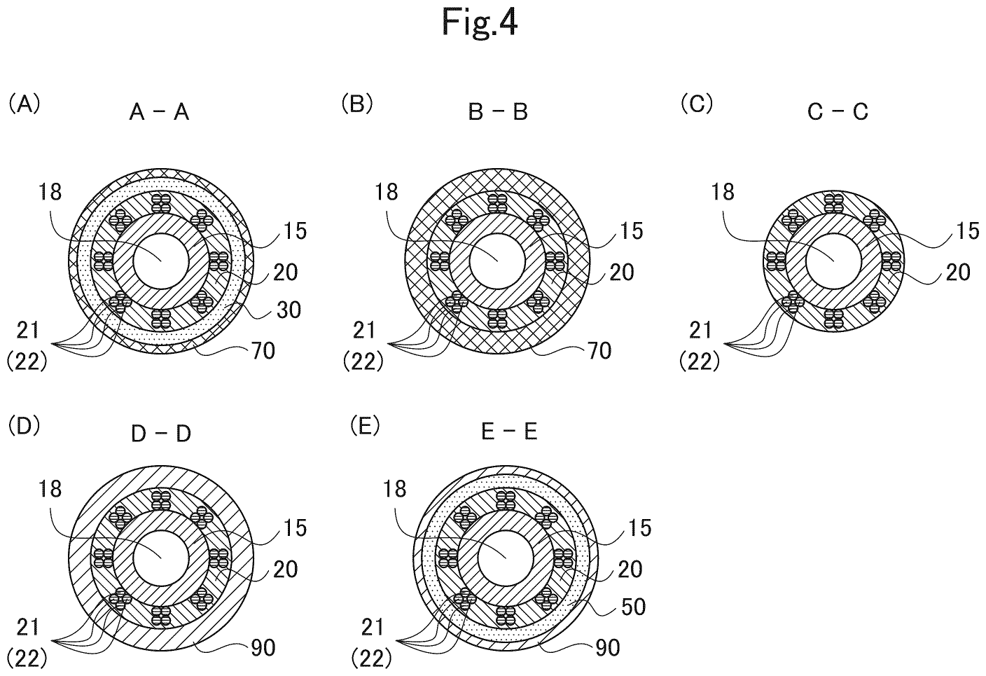

FIG. 4 is an explanatory view illustrating a transverse section of the catheter 1. (A) to (E) of FIG. 4 illustrate the A-A cross-section, the B-B cross-section, the C-C cross-section, the D-D cross-section, and the E-E cross-section of FIG. 2, respectively. Each figure will be described below.
(A) of FIG. 4 illustrates the A-A cross-section of FIG. 2, which is a transverse section of the section S1 (FIG. 3) on the distal end side. As illustrated in (A) of FIG. 4, the outer periphery of the distal end side marker 30 is covered with the distal end side tube 70.
(B) of FIG. 4 illustrates the B-B cross-section of FIG. 2, which is a transverse section of the section S1 (FIG. 3) on the rear end side. As illustrated in (B) of FIG. 4, a portion where the outer periphery of the intermediate tube 20 is covered with the distal end side tube 70 is provided on the rear end side of the rear end 31 (FIG. 3) of the distal end side marker 30.
(C) of FIG. 4 illustrates the C-C cross-section of FIG. 2, which is a transverse section of the section S0 (FIG. 3). As illustrated in (C) of FIG. 4, in the section S0 (FIG. 3), the outer periphery of the intermediate tube 20 is not covered with the distal end side tube 70 or the rear end side tube 90 and is exposed. That is, the gap 80 (FIG. 3) is formed along the outer periphery of the intermediate tube 20.
(D) of FIG. 4 illustrates the D-D cross-section of FIG. 2, which is a transverse section of the section S2 (FIG. 3) on the distal end side. As illustrated in (D) of FIG. 4, a portion where the outer periphery of the intermediate tube 20 is covered with the rear end side tube 90 is provided on the distal end side of the distal end 51 (FIG. 3) of the rear end side marker 50.
(E) of FIG. 4 illustrates the E-E cross-section of FIG. 2, which is a transverse section of the section S2 (FIG. 3) on the rear end side. As illustrated in (E) of FIG. 4, the outer periphery of the rear end side marker 50 is covered with the rear end side tube 90.

The catheter 1 in the section S1 includes the shaft 15, the intermediate tube 20, the distal end side tube 70, and the distal end side marker 30. The catheter 1 in the section S2 includes the shaft 15, the intermediate tube 20, the rear end side tube 90, and the rear end side marker 50. In the catheter 1 in the section S0, the distal end side tube 70, the rear end side tube 90, the distal end side marker 30, and the rear end side marker 50 are not provided. Accordingly, the bending rigidity of the catheter 1 in the section S0 is smaller than the bending rigidity of the catheter 1 in the section S1 and the bending rigidity of the catheter 1 in the section S2. That is, the flexibility of the portion of the catheter 1 between the distal end side tube 70 and the rear end side tube 90 is high. In addition, since the tubes (70, 90) and the markers (30, 50) are not provided at the section S0, the outer diameter of the catheter 1 in the section S0 is smaller than the outer diameter of the catheter 1 in the section S1 or the outer diameter of the catheter 1 in the section S2.

As shown, the braid 21 may be formed by a combination of two wires 22 wound in a first direction and two wires 22 wound in a second direction different from the first direction along the longitudinal direction of the intermediate tube 20. The material of the wire 22 is not particularly limited, and for example, tungsten can be used.

In the catheter 1 according to the first embodiment described above, the rear end 71 of the distal end side tube 70 and the distal end 91 of the rear end side tube 90 are separated from each other. Since the outer layer tubes (70, 90) and the markers (30, 50) are not provided at the section S0, the bending rigidity of the catheter 1 in the section S0 is smaller than the bending rigidity of the catheter 1 in the sections (S1, S2) adjacent to the section S0. In addition, the outer diameter of the catheter 1 in the section S0 is smaller than the outer diameter of the catheter 1 in the sections (S1, S2). Thus, the flexibility of the distal end portion of the catheter 1 can be improved. Since the section S0 is between the distal end side marker 30 and the rear end side marker 50, it is possible to improve the flexibility between the distal end side marker 30 and the rear end side marker 50 in the catheter 1.

The catheter 1 can be used for treatment of an aneurysm. The distal end side marker 30 and the rear end side marker 50 function to indicate the position of the distal end portion of the catheter 1 under X-ray fluoroscopy and can be used for positioning of a delivery device for delivering an embolic substance used for treatment of an aneurysm. Specifically, it is possible to dispose the distal end portion of the delivery device at an appropriate position in the vicinity of the treatment site by confirming that the distal end portion of the catheter 1 is in the vicinity of the treatment site based on the positions of the markers (30, 50) and aligning the positions of the markers (30, 50) with the marker provided at the distal end portion of the delivery device. The portion of the catheter 1 included in the section S is advanced toward the end of the blood vessel along the guide wire inserted into the blood vessel in advance. In some cases, the resistance received from the curved blood vessel, or the like, makes it difficult for the catheter 1 to follow the guide wire and advance in the blood vessel; however, since the portion of the catheter 1 included in the section S is flexible, the catheter 1 can easily follow the guide wire.

The distal end side marker 30 and the rear end side marker 50 are provided at the straight portion 16 of the shaft 15. By providing the markers (30, 50) at the straight portion 16 provided on the distal end side of the catheter 1, it is easy to visually recognize the position of the distal end portion of the catheter 1 inserted into the body under X-ray fluoroscopy.

In the rear end side tube 90, the bending rigidity of the first rear end side tube 92 is smaller than the bending rigidity of the second rear end side tube 93. Thus, the flexibility of the distal end side of the catheter 1 can be improved. The portion (the boundary B0) where the bending rigidity of the rear end side tube 90 changes is between the distal end 17a and the rear end 17b of the tapered portion 17. A change in the bending rigidity of the catheter 1 in the straight portion 16 can be suppressed, and the straight portion 16 can easily follow the guide wire inserted into the body in advance.

The catheter 1 includes the distal tip 110 closer to the distal end side than the distal end side marker 30 and adjacent to the distal end side marker 30. For example, by using a flexible material for the distal tip 110, it is possible to reduce the possibility that the catheter 1 damages the inside of the body.

A part of the shaft 15 is in the gap (the section S0) between the rear end 71 of the distal end side tube 70 and the distal end 91 of the rear end side tube 90. Accordingly, the lumen 18 can be provided even in the section S0, and it is easy to insert another medical device into the body through the lumen 18.

The rear end 71 of the distal end side tube 70 is closer to the rear end side than the rear end 31 of the distal end side marker 30, and the distal end 91 of the rear end side tube 90 is closer to the distal end side than the distal end 51 of the rear end side marker 50. Accordingly, the rear end 31 of the distal end side marker 30 is covered with the distal end side tube 70, and the distal end 51 of the rear end side marker 50 is covered with the rear end side tube 90. Accordingly, the possibility that the markers (30, 50) are exposed to the outside, and that the markers (30, 50) and the body inner wall come into contact with each other, which may damage the body inner wall, may be reduced.

### Second Embodiment

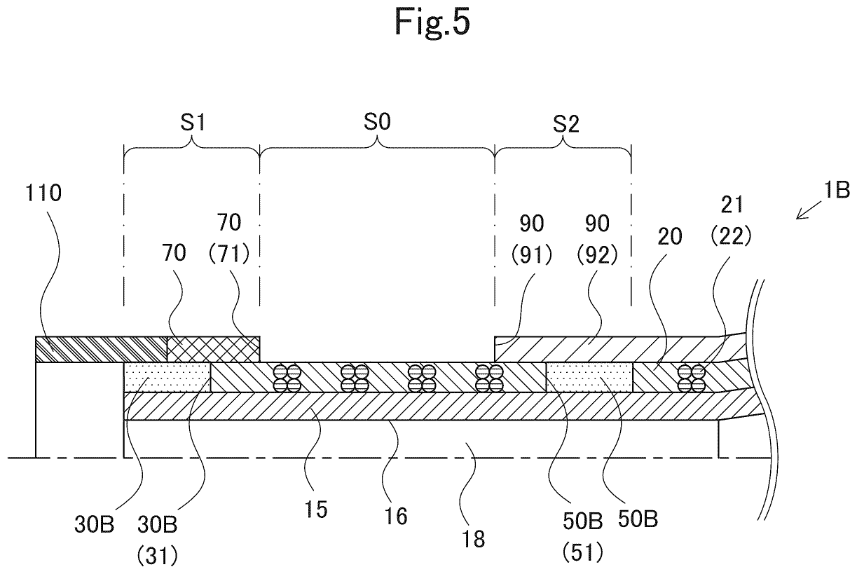

FIG. 5 is an explanatory view illustrating a longitudinal section in the vicinity of markers (30B, 50B) of a catheter 1B according to a second embodiment. The catheter 1B is different from the catheter 1 according to the first embodiment in that the markers (30B, 50B) are embedded in the intermediate tube 20. The description for the configuration of the catheter 1B common to the configuration of the catheter 1 will be omitted.

The distal end side marker 30B and the rear end side marker 50B are embedded in the distal end portion of the intermediate tube 20. A part of the outer periphery of the distal end side marker 30B is covered with the distal end side tube 70. A part of the outer periphery of the rear end side marker 50B is covered with the rear end side tube 90. By embedding the markers (30B, 50B) in the intermediate tube 20, it is easy to reduce the outer diameter of the catheter 1B. The catheter 1B can also improve the flexibility of the distal end portion of the catheter 1B.

### Third Embodiment

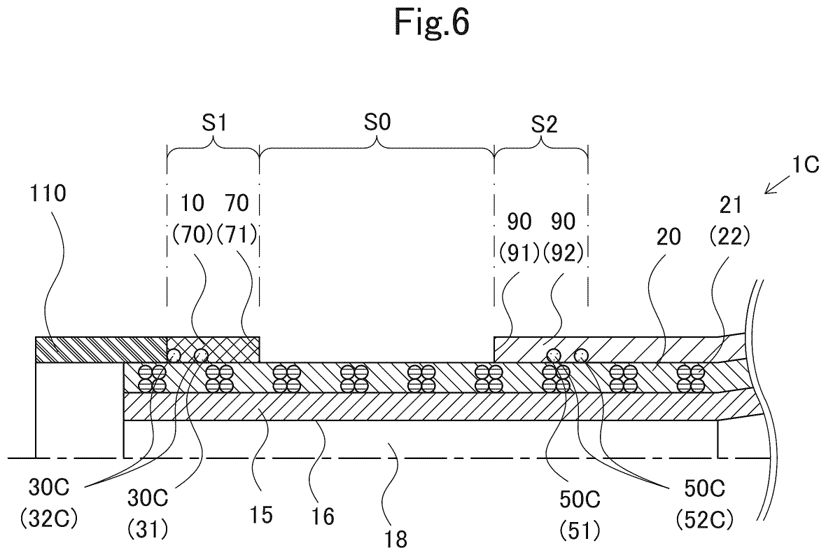

FIG. 6 is an explanatory view illustrating a longitudinal section in the vicinity of markers (30C, 50C) of a catheter 1C according to a third embodiment. The catheter 1C is different from the catheter 1 according to the first embodiment in that the markers (30C, 50C) have a coil shape. The description for the configuration of the catheter 1C common to the configuration of the catheter 1 will be omitted.

The distal end side marker 30C is a coil formed by spirally winding a strand 32C in the longitudinal direction of the catheter 1C. The rear end side marker 50C is a coil formed by spirally winding a strand 52C in the longitudinal direction of the catheter 1C. The strands (32C, 52C) can be made of a radiopaque material, similarly to the markers (30, 50) of the catheter 1 according to the first embodiment described above. The distal end side tube 70 covers the outer periphery of the strand 32C, and the rear end side tube 90 covers the outer periphery of the strand 52C. When the markers (30C, 50C) have a coil shape, an increase in the bending rigidity of the catheter 1C due to the markers (30C, 50C) can be suppressed. The catheter 1C can also improve the flexibility of the distal end portion of the catheter 1C.

### Fourth Embodiment

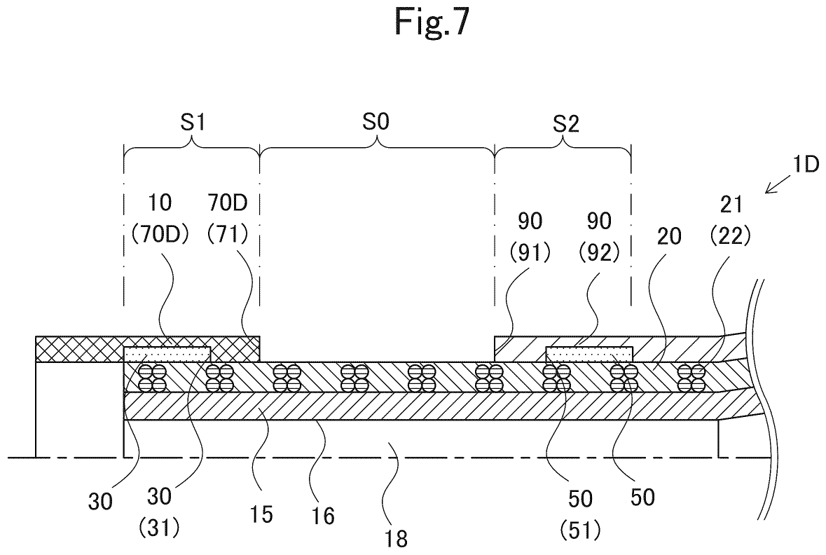

FIG. 7 is an explanatory view illustrating a longitudinal section of the vicinity of the markers (30, 50) of a catheter 1D according to a fourth embodiment. The catheter 1D is different from the catheter 1 according to the first embodiment in that a distal end side tube 70D extends to the distal end side beyond the distal end of the shaft 15. The description for the configuration of the catheter 1D common to the configuration of the catheter 1 will be omitted.

The distal end of the distal end side tube 70D is closer to the distal end side than the distal end of the shaft 15, the distal end of the intermediate tube 20, and the distal end of the distal end side marker 30 in the longitudinal direction of the catheter 1D. The catheter 1D does not include the distal tip 110 included in the catheter 1 according to the first embodiment, but the distal end portion of the distal end side tube 70D can have the same function as the distal tip 110. That is, by using a flexible material for the distal end side tube 70D, it is possible to reduce the possibility that the catheter 1D damages the inside of the body. In addition, since there is no possibility that the distal tip 110 and the distal end side tube 70D are broken at the connection portion between the distal tip 110 and the distal end side tube 70D, the durability of the catheter 1D can be improved. The catheter 1D can also improve the flexibility of the distal end portion of the catheter 1D.

### Fifth Embodiment

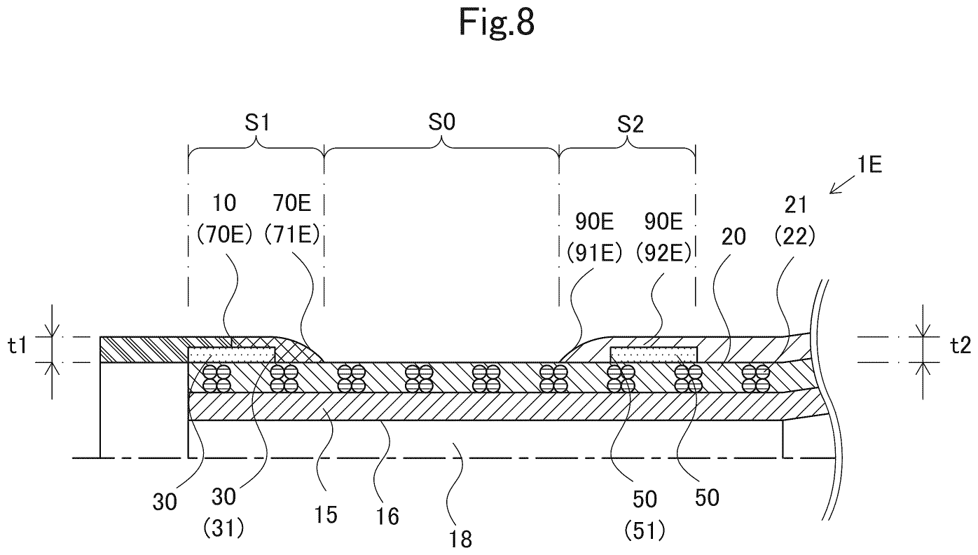

FIG. 8 is an explanatory view illustrating a longitudinal section of the vicinity of the markers (30, 50) of a catheter 1E according to a fifth embodiment. The catheter 1E is different from the catheter 1 according to the first embodiment in that the shapes of a rear end 71E of a distal end side tube 70E and a distal end 91E of a rear end side tube 90E (a first rear end side tube 92E) are a tapered shape. The description for the configuration of the catheter 1E common to the configuration of the catheter 1 will be omitted.

According to the present embodiment, the thickness of the distal end side tube 70E is referred to as a “thickness t1”, and the thickness of the rear end side tube 90E is referred to as a “thickness t2”. The rear end 71E of the distal end side tube 70E has a tapered shape in which the thickness t1 gradually decreases from the distal end side toward the rear end side of the catheter 1E. The distal end 91E of the rear end side tube 90E has a tapered shape in which the thickness t2 gradually decreases from the rear end side toward the distal end side of the catheter 1E. A part of the outer peripheral surface of the rear end 71E of the distal end side tube 70E and the distal end 91E of the rear end side tube 90E is formed by a curved surface. Accordingly, the bending rigidity in the longitudinal direction of the catheter 1E can gently change in the vicinity of the rear end 71E of the distal end side tube 70E and in the vicinity of the distal end 91E of the rear end side tube 90E. The catheter 1E can also improve the flexibility of the distal end portion of the catheter 1E.

### Modification

The disclosed embodiments are not limited to the above-described embodiment and can be implemented in various modes without departing from the spirit of the present disclosure, and for example, the following and other modifications are also possible.

### Modification 1

In the catheters (1, 1B, 1C, 1D, 1E) according to the first embodiment to the fifth embodiment, the distal end side markers (30, 30B, 30C) and the rear end side markers (50, 50B, 50C) are provided at the straight portion 16 of the shaft 15. However, the distal end side markers (30, 30B, 30C) and the rear end side markers (50, 50B, 50C) may be provided at the tapered portion 17 of the shaft 15.

### Modification 2

The catheters (1, 1B, 1C, 1D, 1E) according to the first embodiment to the fifth embodiment are configured by overlapping the shaft 15, the intermediate tube 20, the distal end side tube (70, 70D, 70E), and the rear end side tube (90, 90E). However, the catheters (1, 1B, 1C, 1D, 1E) do not need to include the shaft 15 and do not need to include the intermediate tube 20. For example, the catheters (1, 1B, 1C, 1D, 1E) may be configured by the distal end side tube (70, 70D, 70E) that covers the outer periphery of the distal end side marker (30, 30B, 30C) and the rear end side tube (90, 90E) that covers the outer periphery of the rear end side marker (50, 50B, 50C).

### Modification 3

In the catheters (1, 1B, 1C, 1D, 1E) according to the first embodiment to the fifth embodiment, the rear end (71, 71E) of the distal end side tube (70, 70D, 70E) is closer to the rear end side than the rear end 31 of the distal end side marker (30, 30B, 30C), and the distal end (91, 91E) of the rear end side tube (90, 90E) is closer to the distal end side than the distal end 51 of the rear end side marker (50, 50B, 50C). However, the rear end (71, 71E) of the distal end side tube (70, 70D, 70E) may be closer to the distal end side than the rear end 31 of the distal end side marker (30, 30B, 30C), and the distal end (91, 91E) of the rear end side tube (90, 90E) may be closer to the rear end side than the distal end 51 of the rear end side marker (50, 50B, 50C).

### [Aspects]

(1) According to an aspect, a catheter includes a hollow shaft, a distal end side marker that is provided at a distal end portion of the shaft, a rear end side marker that is provided at the distal end portion of the shaft and is closer to a rear end side than the distal end side marker, a distal end side tube covering an outer periphery of the distal end side marker, and a rear end side tube covering an outer periphery of the rear end side marker, and a rear end of the distal end side tube and a distal end of the rear end side tube are provided to be separated from each other.

With this configuration, since the distal end side tube and the rear end side tube are separated from each other, the flexibility of the distal end portion of the catheter can be improved.

(2) In the catheter according to the above-described aspect, the shaft may include a straight portion having a substantially constant outer diameter in an axial direction of the shaft, and a tapered portion that is provided closer to a rear end side than the straight portion and has a larger outer diameter at a rear end than an outer diameter at a distal end, and the distal end side marker and the rear end side marker may be provided at the straight portion.

With this configuration, since the distal end side marker and the rear end side marker are provided at the straight portion, it is easy to visually recognize the position of a portion closer to the distal end in the catheter inserted into the body under X-ray fluoroscopy.

(3) In the catheter according to the above aspect, the rear end side tube may cover an outer periphery of the tapered portion, and a bending rigidity of a portion of the rear end side tube that covers a distal end side of the tapered portion may be smaller than a bending rigidity of a portion of the rear end side tube that covers a rear end side of the tapered portion.

With this configuration, since the bending rigidity of the portion closer to the distal end side in the rear end side tube is small, it is possible to improve the flexibility of the distal end side of the catheter.

(4) The catheter according to the above-described aspect may further include a distal tip that is closer to a distal end side than the distal end side marker and is adjacent to the distal end side marker.

With this configuration, for example, by forming the distal tip with a flexible material, it is possible to improve the flexibility of the most distal end of the catheter.

(5) In the catheter according to the above aspect, a part of the shaft may be provided between the rear end of the distal end side tube and the distal end of the rear end side tube.

With this configuration, since the shaft is provided between the distal end side tube and the rear end side tube, it is easy to advance another medical device into the body through the inside of the catheter.

(6) In the catheter according to the above aspect, the rear end of the distal end side tube may be closer to a rear end side than a rear end of the distal end side marker, and the distal end of the rear end side tube may be closer to a distal end side than a distal end of the rear end side marker.

With this configuration, the distal end side tube covers the rear end of the distal end side marker, and the rear end side tube covers the distal end of the rear end side marker, so that it is possible to reduce the possibility that the markers are exposed.

The present invention can be implemented in various aspects and can be implemented in the form of, for example, a guide wire, a method for manufacturing a guide wire, a catheter, a method for manufacturing a catheter, an endoscope, or a dilator.

### DESCRIPTION OF REFERENCE NUMERALS

1 CATHETER
15 SHAFT
16 STRAIGHT PORTION
17 TAPERED PORTION
18 LUMEN
20 INTERMEDIATE TUBE
21 BRAID
22 WIRE
30 DISTAL END SIDE MARKER
31 REAR END OF DISTAL END SIDE MARKER
50 REAR END SIDE MARKER
51 DISTAL END OF REAR END SIDE MARKER
70 DISTAL END SIDE TUBE
71 REAR END OF DISTAL END SIDE TUBE
90 REAR END SIDE TUBE
91 DISTAL END OF REAR END SIDE TUBE
110 DISTAL TIP
120 CONNECTOR
S0 SECTION BETWEEN DISTAL END SIDE TUBE AND REAR END SIDE TUBE
S1 SECTION FROM DISTAL END OF DISTAL END SIDE MARKER TO REAR END OF DISTAL END SIDE TUBE
S2 SECTION FROM REAR END OF REAR END SIDE MARKER TO DISTAL END OF REAR END SIDE TUBE

What is claimed is:

1. A catheter comprising:
a hollow shaft;
a distal end side marker at a distal end portion of the shaft;
a rear end side marker at the distal end portion of the shaft and closer to a rear end side than the distal end side marker;
a distal end side tube covering an outer periphery of the distal end side marker; and
a rear end side tube covering an outer periphery of the rear end side marker, wherein
a rear end of the distal end side tube and a distal end of the rear end side tube are separated from each other.

2. The catheter according to claim 1, wherein
the shaft includes
a straight portion having a constant outer diameter in an axial direction of the shaft, and
a tapered portion closer to a rear end side than the straight portion and having a larger outer diameter at a rear end than an outer diameter at a distal end, wherein
the distal end side marker and the rear end side marker are at the straight portion.

3. The catheter according to claim 2, wherein
the rear end side tube covers an outer periphery of the tapered portion, and
a bending rigidity of a portion of the rear end side tube that covers a distal end side of the tapered portion is smaller than a bending rigidity of a portion of the rear end side tube that covers a rear end side of the tapered portion.

4. The catheter according to claim 3, further comprising a distal tip closer to a distal end side than the distal end side marker and adjacent to the distal end side marker.

5. The catheter according to claim 3, wherein a part of the shaft is between the rear end of the distal end side tube and the distal end of the rear end side tube.

6. The catheter according to claim 3, wherein
the rear end of the distal end side tube is closer to a rear end side than a rear end of the distal end side marker, and
the distal end of the rear end side tube is closer to a distal end side than a distal end of the rear end side marker.

7. The catheter according to claim 1, wherein
the rear end side tube covers an outer periphery of the tapered portion, and
a bending rigidity of a portion of the rear end side tube that covers a distal end side of the tapered portion is smaller than a bending rigidity of a portion of the rear end side tube that covers a rear end side of the tapered portion.

8. The catheter according to claim 7, further comprising a distal tip closer to a distal end side than the distal end side marker and adjacent to the distal end side marker.

9. The catheter according to claim 8, wherein a part of the shaft is between the rear end of the distal end side tube and the distal end of the rear end side tube.

10. The catheter according to claim 8, wherein
the rear end of the distal end side tube is closer to a rear end side than a rear end of the distal end side marker, and
the distal end of the rear end side tube is closer to a distal end side than a distal end of the rear end side marker.

11. The catheter according to claim 1, further comprising a distal tip closer to a distal end side than the distal end side marker and adjacent to the distal end side marker.

12. The catheter according to claim 11, wherein a part of the shaft is between the rear end of the distal end side tube and the distal end of the rear end side tube.

13. The catheter according to claim 12, wherein
the rear end of the distal end side tube is closer to a rear end side than a rear end of the distal end side marker, and
the distal end of the rear end side tube is closer to a distal end side than a distal end of the rear end side marker.

14. The catheter according to claim 1, wherein a part of the shaft is between the rear end of the distal end side tube and the distal end of the rear end side tube.

15. The catheter according to claim 14, wherein
the rear end of the distal end side tube is closer to a rear end side than a rear end of the distal end side marker, and
the distal end of the rear end side tube is closer to a distal end side than a distal end of the rear end side marker.

16. The catheter according to claim 1, wherein
the rear end of the distal end side tube is closer to a rear end side than a rear end of the distal end side marker, and
the distal end of the rear end side tube is closer to a distal end side than a distal end of the rear end side marker.
